(19) United States
(12) Patent Application Publication
(10) Pub. No.: US 2012/0209357 A1
(43) Pub. Date: Aug. 16, 2012

(54) PORTABLE SKIN TREATMENT DEVICE USING LIGHT AND HEAT AND METHOD OF CONTROLLING THE SAME

(75) Inventor: Donghoon HA, Seoul (KR)

(73) Assignee: MEDICON CO., LTD., Gangwon-do (KR)

(21) Appl. No.: 13/026,343

(22) Filed: Feb. 14, 2011

(51) Int. Cl. A61N 5/06; A61F 7/08

(52) U.S. Cl. 607/90

(57) ABSTRACT

A method of controlling an operation of a portable skin treatment device, the method including: if a control button of the treatment device is pressed, heating a heater to prepare heating of a tip of the treatment device in a predetermined temperature range; and if heating of the tip is completed, turning on a light source disposed inside a housing of the treatment device and performing light irradiation treatment of irradiating light onto the skin through the tip together with heat. By using the method of controlling the operation of the portable skin treatment device by applying heat to the skin area of a disease of sebaceous glands such as acne etc. or itch and by irradiating light onto the skin area, treatment effects can be improved, and the fact that a user is getting treatment can be more realistically recognized.

### BACKGROUND OF THE INVENTION

1. Field of the Invention

The present invention relates to a skin treatment device by applying heat to the skin and a method of controlling the same, and more particularly, to a portable skin treatment device.

2. Description of the Related Art

Technology for treatment of skin inflammation, skin itch and other skin diseases by applying heat of a predetermined temperature to the skin is known. There is a representative example thereof, disclosed in U.S. Application Publication No. US 2001/0008974 entitled “Method and apparatus for treatment of skin itch and disease”. In the related art, skin treatment effects are achieved by applying heat of a temperature of 49-62° C. to an area of a skin disease. Furthermore, the related art suggests a portable skin treatment apparatus powered by a self-contained battery.

Unlike special treatment devices used in a dermatology clinic, since portable devices using power of a self-contained battery are generally low-priced, the range of customers using the devices increases. Furthermore, since the devices are portable, they can be more freely used without the limitation in places and time. Thus, when treatment by a special medicine or a precise treatment device is not necessarily required, the portable treatment devices will be helpful to ordinary patients, in particular.

For example, skin diseases such as acne that is common in adolescents, itch such as athlete's foot, itch when a man is bitten by the insect such as mosquito, occur frequently in a daily life. When symptoms of a patient are severe, it is recommended that the patient gets treatment in a clinic. However, when symptoms of a patient are not severe and a skin disease is a usual disease, it will be best not to go to the clinic but to do treatment on site immediately for himself/herself. For example, when a man is bitten by mosquito and has an itch, a man does not need to go to the clinic. Thus, the use of portable treatment devices will be a worldwide trend.

However, since the portable treatment devices are portable devices that a patient uses for himself/herself, it is difficult to say that treatment effects thereof are more excellent than those in the clinic. This is because there are limitations such as power consumption of the portable treatment devices, limitations in using time, non-combination with special treatment methods, and the like. Due to the problems, many efforts for improving treatment effects have been made in the field of the portable treatment devices. In detail, the efforts for improving the performance of portable devices that may achieve treatment effects by the application of heat have been made.

When a doctor treats a patient by using a treatment device, the patient is not concerned about whether he/she is getting treatment or not. This is because it is a matter of course that he/she goes to the clinic and thus gets treatment. However, when the patient wants to treat himself/herself by using a portable treatment device, the patient wants to know whether the portable treatment device operates properly and he/she is getting treatment accurately. However, in the case of a treatment method using heat shock, a user cannot easily check whether the user gets treatment by the treatment method using heat shock. Since a treatment means in a heat shock technique depends on heat of a predetermined temperature and a time variable for applying heat, it is difficult to easily check whether heat that the user feels is warm-up heat to be used to perform a heat treatment operation, heat applied during the operation, or heat that remains after the operation is completed.

### SUMMARY OF THE INVENTION

The present invention provides a portable skin treatment device having an improved structure for treating skin diseases such as skin itch that occurs due to athlete's foot or when a man is bitten by the insect such as mosquito, acne, and the like, and a method of controlling the same.

The present invention also provides a portable skin treatment device having an improved structure that achieves improved treatment effects compared to those of a related art heat applying method by applying heat to the area of a skin disease and by irradiating light in a predetermined wavelength range onto the area of the skin disease, and a method of controlling the same. In other words, an aspect of treatment of the present invention may be classified into treatment using heat and treatment using light, and the present invention improves treatment effects by combining treatment using heat and treatment using light.

The present invention also provides a portable skin treatment device having an improved structure that secures visibility of treatment. Since not professional but a patient operates the portable skin treatment device for himself/herself so as to use it, a user has a desire to check for himself/herself whether the user is getting treatment or the portable skin treatment device operates properly. The present invention is provided to satisfy the user's desire. The objective of the present invention has a meaning that the present invention is a new and inventive product based on new technology for combining two treatment methods.

The present invention also provides a portable skin treatment device having an improved structure in which a treatment device can be used using a battery that a user can easily purchase so as to avoid inconvenience in time required for charging or loss or failure of a charging device.

Meanwhile, unspecified other objectives of the present invention will be additionally considered within the range where they can be easily inferred from the following detailed description and effects thereof.

According to an aspect of the present invention, there is provided a method of controlling an operation of a portable skin treatment device, the method comprising:

heating a heater to prepare heating of a tip of the treatment device in a predetermined temperature range, if a control button of the treatment device is pressed; and

turning on a light source disposed inside a housing of the treatment device and performing light irradiation treatment of irradiating light onto the skin through the tip together with heat, if heating of the tip is completed,

wherein the light source is a blue light emitting diode (LED), a red LED, an LED having red, green, and blue (RGB) color, or an LED that optionally emits blue or red light, and

when the light irradiation treatment is performed together with heat, a temperature of the tip is 47.2 to 49.4° C., and the time when the light irradiation treatment is performed together with heat is 120 to 180 sec.

According to another aspect of the present invention, there is provided a method of controlling an operation of a portable skin treatment device, the method comprising:

determining an input of a control button of the treatment device;

turning on a light source disposed inside a housing of the treatment device and irradiating light onto the skin through the tip together with heat, if the input is performed;

heating a heater so that a temperature of a tip of the treatment device reaches a predetermined temperature, while the light irradiation treatment is performed; and

outputting a heating completion signal to a display unit of the treatment device if heating is completed, and performing heat treatment in addition to the light irradiation treatment,

wherein the light source is a blue light emitting diode (LED), a red LED, an LED having red, green, and blue (RGB) color, or an LED that optionally emits blue or red light, and

when the light irradiation treatment is performed together with the heat treatment, a temperature of the tip is 47.2 to 49.4° C., and the time when the light irradiation treatment is performed together with the heat treatment is 120 to 180 sec.

According to another aspect of the present invention, there is provided a portable skin treatment device that operates by using the above-described method, the portable skin treatment device including:

a housing which comprises a control button, a display unit, and a tip for skin contact and in which a printed circuit board (PCB) is disposed,

wherein internal circuit elements are mounted on the PCB, the internal circuit elements comprising:
a heater heating the tip and a heater controller controlling the heater;
a light source irradiating light onto an outside of the treatment device through the tip;
a microcomputer controller controlling an operation of the treatment device; and
a power supply unit supplying power, and
wherein the light source is attached to an edge surface of an end of the PCB that contacts the tip.

According to another aspect of the present invention, there is provided a portable skin treatment device in which a printed circuit board (PCB) on which an internal circuit unit is mounted, is inserted in a housing having a hollow tube shape, the portable skin treatment device comprising:

a tip for skin contact installed on one end of the housing;

a battery cap installed on the other end of the housing, sealing the housing and simultaneously electrically contacting a terminal inside the housing;

a battery unit in which two alkaline batteries having a specification of AAA or AA and an electromotive force of 1.5 V are inserted in the housing; and

a control button and a display unit installed on a surface of the housing,

wherein internal circuit elements are mounted on the PCB, the internal circuit elements including:
a heater heating the tip and a heater controller controlling the heater;
a light source irradiating light being a light emitting diode (LED) attached to an edge surface of the end of the PCB that contacts the tip and irradiating light onto an outside of the treatment device through the tip; and
a microcomputer controller receiving an input of the control button, controlling an operation of the treatment device and displaying an operating state on the display unit,
wherein heat treatment and light irradiation treatment are performed by the input of the control button.

### BRIEF DESCRIPTION OF THE DRAWINGS

The above and other features and advantages of the present invention will become more apparent by describing in detail exemplary embodiments thereof with reference to the attached drawings in which:

FIGS. 1 and 2 are perspective views illustrating an example of a housing of a portable skin treatment device 100 according to an embodiment of the present invention;

FIGS. 3A and 3B illustrate examples of charging devices of the portable skin treatment device 100 illustrated in FIGS. 1 and 2, and FIG. 3C illustrates a state where a jack of a charging device is inserted in the portable skin treatment device 100;

FIG. 4 is an enlarged perspective view of a configuration example of an operating unit having a tip 10 for skin contact of the portable skin treatment device 100 illustrated in FIGS. 1 and 2;

FIGS. 5 and 6 are perspective views illustrating an example of a housing of a portable skin treatment device 100 according to another embodiment of the present invention;

FIG. 7 illustrates a state where two alkaline batteries are inserted in the housing of the portable skin treatment device 100 illustrated in FIGS. 5 and 6;

FIG. 8 is an enlarged perspective view of a configuration example of an operating unit having a tip for skin contact of the portable skin treatment device 100 illustrated in FIGS. 5 and 6;

FIG. 9 illustrates a state where a printed circuit board (PCB) 250 and a tip 10 for skin contact are fixed in the housing of the portable skin treatment device illustrated in FIGS. 1 and 2 and FIGS. 5 and 6;

FIGS. 10 and 11 are schematic circuit diagrams of the portable skin treatment device 100 illustrated in FIGS. 1 and 2 and FIGS. 5 and 6;

FIG. 12 are graphs showing wavelength of light to be irradiated by the portable skin treatment device 100 and a light irradiation method, according to an embodiment of the present invention;

FIG. 13 is a graph showing heating versus temperature of the portable skin treatment device 100, according to an embodiment of the present invention;

FIG. 14 illustrates a state where the portable skin treatment device 100 illustrated in FIGS. 1 and 2 and FIGS. 5 and 6 is used;

FIG. 15 are images showing treatment effects of the portable skin treatment device 100, based on FIGS. 12 and 13; and

FIGS. 16 through 18 are flowcharts illustrating various control processes of an operation of the portable skin treatment device 100.

### DETAILED DESCRIPTION OF THE INVENTION

The present invention will now be described more fully with reference to the accompanying drawings, in which exemplary embodiments of the invention are shown. In the description of the present invention, if it is determined that a detailed description of commonly-used technologies or structures related to the invention may unnecessarily obscure the subject matter of the invention, the detailed description will be omitted.

### First Embodiment

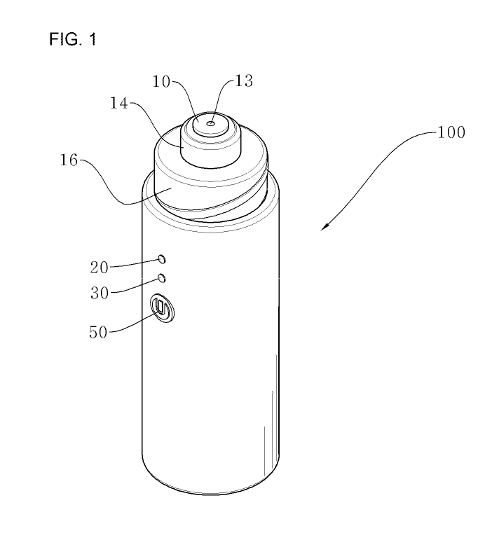

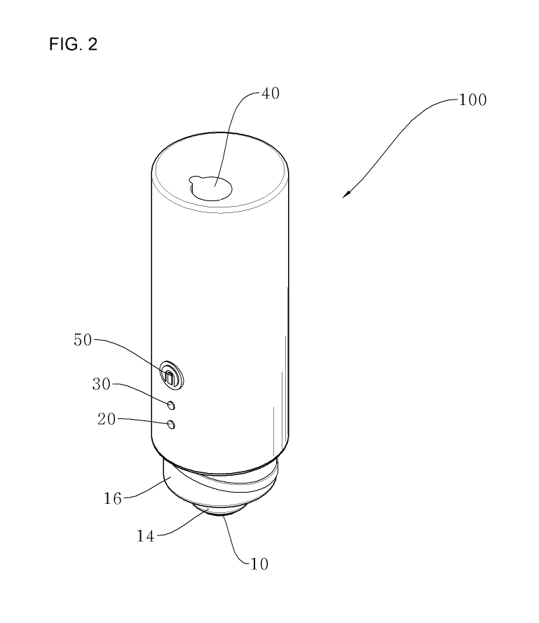

FIGS. 1 and 2 are perspective views illustrating an example of an external structure of a portable skin treatment device 100 according to an embodiment of the present invention. A housing of the portable skin treatment device 100 may be cylindrical to be easily carried by a user and to be easily grasped by hands. A control button 50 that is a user interface and one or more display lamps 20 and 30 may be formed on an outer circumferential surface of the housing. The two display lamps 20 and 30 may be lamps that emit light having different colors, for example, green and blue, so that the user easily differentiates two treatment effects according to the present invention from each other. The display lamps 20 and 30 may be light emitting diodes (LEDs).

A tip 10 for skin contact is formed on an end of the housing that contacts the skin, and a tip fixing portion 14 for fixing the tip 10 in the housing, and a cap combining portion 16 that combines with a cap (not shown) for protecting the tip 10 in a screw manner are formed on the housing. In another embodiment, the cap may be attached to or detached from the cap combining portion 16 of the housing of the portable skin treatment device in a sliding manner. Since the tip 10 may be attached to one end of a printed circuit board (PCB) installed in the portable skin treatment device, the tip 10 may be fixed in the housing by using the tip fixing portion 14 and simultaneously, the PCB may also be fixed in the housing.

A length of the housing of the portable skin treatment device 100 may be about 60 to 80 mm, and a diameter thereof may be 22 to 26 mm. However, the length and diameter of the housing may be changed in various sizes according to configuration of an internal circuit unit, the type of battery, a design factor, or the like. However, the sizes of the length and diameter of the housing may be easily carried and grasped by the user. Material used to form the housing may be an acrylonitrile butadiene styrene (ABS) resin that is easily processed and has a good impact resistance and a good thermal resistance.

The tip 10 transfers heat to the skin and thus may be formed of a component having good thermal conductivity. The tip 10 may be formed of metal or non-metal having good thermal conductivity, for example. When the tip 10 is formed of metal, gold (Au), silver (Ag), aluminum (Al), tungsten (W), copper (Cu), or the like may be used to form the tip 10. However, in consideration of an economical aspect, Al having a thickness of 0.5 mm may be used. When the tip 10 is formed of non-metal, thermal conductive plastics, fine ceramics such as aluminum nitride (AlN), or the like may be used.

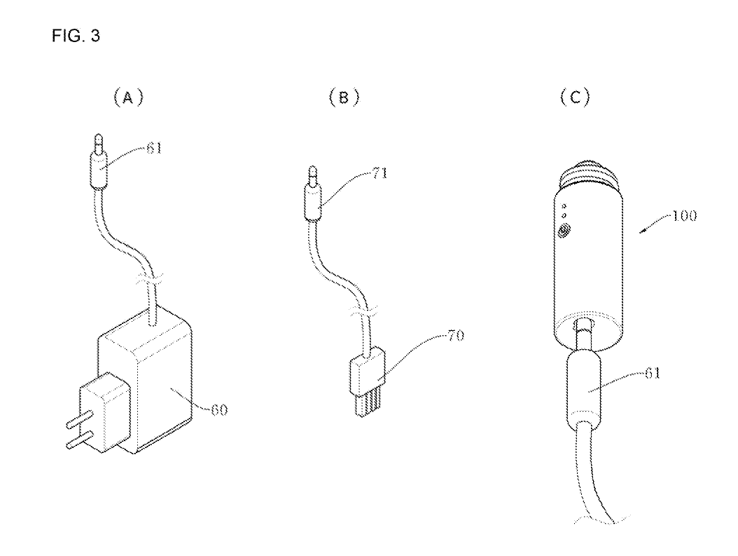

As illustrated in FIG. 2, a charging hole 40 in which a self-contained battery can be charged may be formed on an opposite end of the housing to the end of the housing that contacts the skin. One of charging jacks 61 and 71 of each of charging devices 60 and 70 to be separately provided may be inserted in the charging hole 40 so as to charge the self-contained battery. As illustrated in FIG. 3A, the charging device 60 may be an adaptor that outputs a voltage of 5V by putting a plug in a commonly-used power supply of 100 to 240 V so as to supply the voltage of 5V to the portable skin treatment device 100. Also, as illustrated in FIG. 3B, the charging device 70 may be a universal serial bus (USB) cable charged using a USB voltage of a computer. As illustrated in FIG. 3C, one of the charging jacks 61 and 71 of each of the charging devices 60 and 70 is inserted in the charging hole 40 of the portable skin treatment device 100 so as to charge the self-contained battery. Power supplies of the charging devices 60 and 70 charge the self-contained battery through an internal charging circuit. In this regard, a microcomputer controller 111 may display a charging state to the user by using the two display lamps 20 and 30. For example, when charging starts, the green lamp 20 that emits green light is turned on, and when charging is completed, the blue lamp 30 that emits blue light may be turned on.

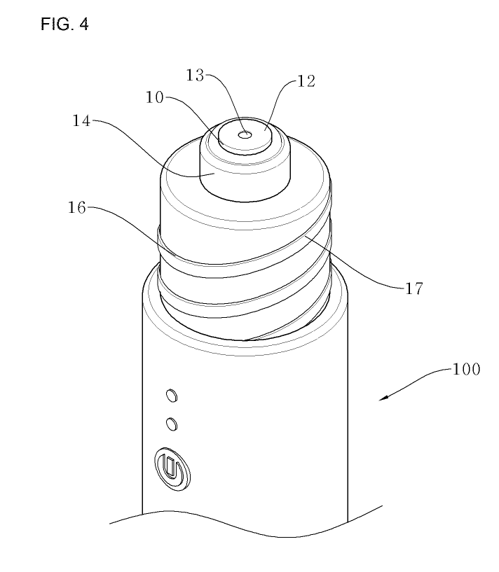

FIG. 4 is an enlarged perspective view of configuration of an upper portion of the housing of the portable skin treatment device 100, i.e., the end of the housing that contacts the skin. First, a protrusion of the cap combining portion 16 wound around the cap combining portion 16 in an oblique line direction is a screw thread to be combined with the cap. The skin contact a surface 12 of the tip 10 for skin contact that protrudes from the tip fixing portion 14, and thus, heat from the heated tip 10 is applied to the skin. The tip 10 has a hollow cylindrical shape, and a light source (not shown) may be disposed inside the tip 10. A light irradiation hole 13 may be formed in the center of the surface 12 of the tip 10. Light emitted from the light source may be irradiated onto an outside of the surface 12 of the tip 10 through the light irradiation hole 13.

Considering the light source is disposed in the housing of the portable skin treatment device 100 and a visible light transmission distance of a built-in LED as a light source is just several centimeters, in order to improve light irradiation effects, the light source may be disposed most adjacent to the surface 12 of the tip 10. However, in the case of a laser pointer that transmits visible light to a farther distance, a long light irradiation path may be formed in the housing, and a laser light source may be disposed at an inner position of the housing.

### Second Embodiment

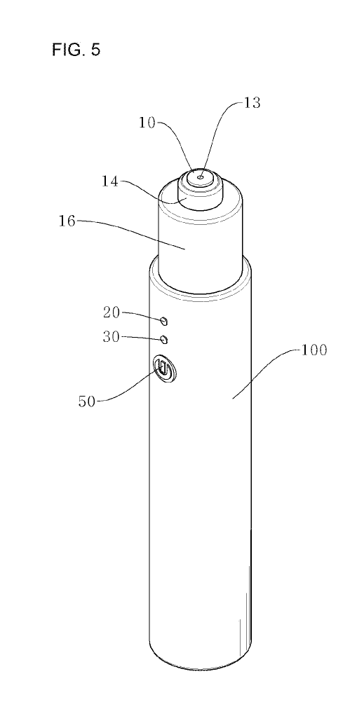

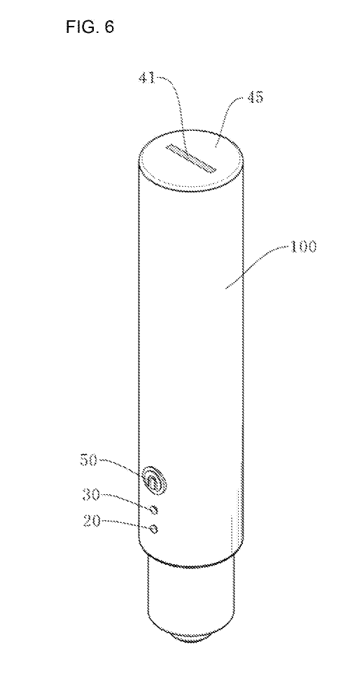

FIGS. 5 and 6 are perspective views illustrating an example of an external structure of a portable skin treatment device 100 according to another embodiment of the present invention. The portable skin treatment device 100 illustrated in FIGS. 5 and 6 has a structure of a longer and thinner housing than that of the portable skin treatment device 100 illustrated in FIGS. 1 and 2. However, materials used to form the housing and a tip of the portable skin treatment device 100 illustrated in FIGS. 5 and 6 are the same as those of the portable skin treatment device 1000 illustrated in FIGS. 1 and 2.

In the current embodiment, a length of the housing of the portable skin treatment device 100 may be about 140 to 160 mm, and a diameter thereof may be 14 to 20 mm. However, the length and diameter of the housing may be changed in various sizes according to configuration of an internal circuit unit, the type of battery, a design factor, or the like.

For example, when an alkaline battery having a specification of AAA is built in the housing, a diameter of a cross-section of the housing may be about 15 mm. However, in the case of an alkaline battery having a specification of AA, a diameter thereof may be further increased. Also, when two alkaline batteries are built in the housing, a better output performance can be achieved compared to a case where one alkaline battery is built in the housing, but the length of the housing is increased. When one alkaline battery is built in the housing, the length of the housing may be about 70 mm. Various modifications of the specification of the size of the housing may be possible. However, it will be emphasized that the size of the housing may be easily carried and grasped by the user.

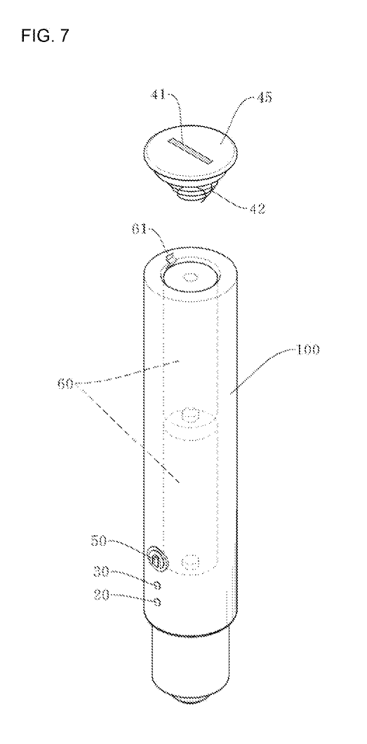

As illustrated in FIG. 7, a battery 60 may be inserted in the housing through an opposite end of the housing to the end of the housing that contacts the skin. The battery may be an alkaline battery having an electromotive force of 1.5 V. In order to make the housing thinner and simultaneously to easily produce a desired output performance, two alkaline batteries having the specification of 1.5 V AAA may be inserted in the housing in series. Two alkaline batteries having the specification of 1.5 V AA may be inserted in the housing. However, the diameter of the cross-section of the housing may be increased as the specification of the battery is increased. Since a change of the size of the housing is determined by a design factor, the protection scope of the present invention is not limited by an AA or AAA specification.

In FIG. 6, two alkaline batteries having the specification of AAA are inserted in the order of +/− and are sealed by a battery cap 45. The battery cap 45 has a contact portion 42 that electrically contacts the inserted battery, and the contact portion 42 electrically contacts a terminal inside the housing, thereby supplying power to an internal circuit of the portable skin treatment device 100. The battery cap 45 may be connected to the end of the housing in a screw combining method or may be disconnected therefrom. Also, for convenience of connection and disconnection, a groove 41 may be formed in the battery cap 45. This is as illustrated in FIG. 7.

Even when the battery 60 is inserted, if the portable skin treatment device 100 does not operate, a supply of power is cut off. When a control button 50 installed on the surface of the housing is pressed, the supply of power starts. In this regard, the microcomputer controller 111 may display a charging state to the user by using the two display lamps 20 and 30 that the supply of power is on and heating by a heater starts. For example, if the control button 50 is pressed, the green lamp 20 may be turned on, and if the control button is pressed for a long time in the state where the green lamp 20 is turned on, the supply of power may be off, and the green lamp 20 may be turned off. Here, both the green lamp 20 and the blue lamp 30 may be turned off.

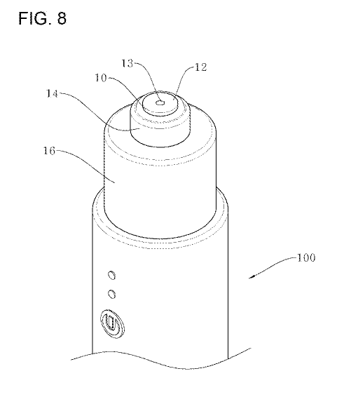

FIG. 8 is an enlarged perspective view of a configuration example of an operating unit having a tip for skin contact of a portable skin treatment device 100 illustrated in FIGS. 5 and 6. First, an inside of a cap (not shown) closely contacts a cam combining portion 16 and is slid so that the cap and the portable skin treatment device 100 are detached from each other. The skin contacts a surface 12 of a tip for skin contact 10 that protrudes from a tip fixing portion 14, and thus, heat from the heated tip 10 is transferred to the skin. The tip 10 has a hollow cylindrical shape, and a light source (not shown) may be disposed inside the tip 10. A light irradiation hole 13 may be formed in the center of the surface 12 of the tip 10. Light emitted from the light source may be irradiated onto an outside of the surface 12 of the tip 10 through the light irradiation hole 13.

Considering the light source is disposed in the housing of the portable skin treatment device 100 and a visible light transmission distance of a built-in LED as a light source is just several centimeters, in order to improve light irradiation effects, the light source may be disposed most adjacent to the surface 12 of the tip 10. However, in the case of a laser pointer that transmits visible light to a farther distance, a long light irradiation path may be formed in the housing, and a laser light source may be disposed at an inner position of the housing.

### <Internal Configuration of Portable Skin Treatment Device 100>

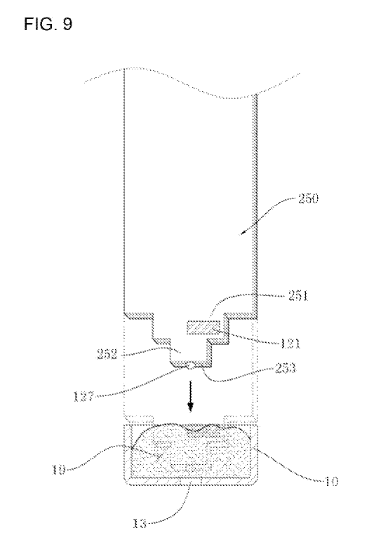

FIG. 9 illustrates an internal configuration of the tip 10 for skin contact of the portable skin treatment device 100 illustrated in FIGS. 1 and 2 and FIGS. 5 and 6, and the relationship between light and heat in detail. Internal circuit elements are mounted on a printed circuit board (PCB) 250 having an approximately rectangular shape to be built in a housing but are omitted in the drawings for convenience of explanation.

A width of an end of the PCB 250 that contacts a tip 10 may be reduced in a step manner. A heater 121 formed of a resistor may be disposed at a front portion 251 of the PCB 250. Also, a predetermined temperature sensor and a heating control circuit may be disposed adjacent to each other. A front end portion 252 of the PCB 250 may be inserted in the tip 10. A light source 127 such as a light emitting diode (LED) is attached to an edge surface 253 of the front end portion 252 inserted in the tip 10 so that the light source 127 is most adjacent to a surface 12 of the tip 10. In this way, the LED light source 127 is attached to the edge surface 253 of the front end portion 252 of the PCB 250 so that a light irradiation direction of the LED light source 127 is identical with a user contact direction of the tip 10 and efficiency of light irradiation treatment is improved.

The LED light source 127 may be a blue LED, a red LED, or an LED that emits blue or red light alternately. Also, the LED light source 127 may be a red, green, and blue (RGB) LED that optionally emits blue light and red light. In particular, in the case of an LED that optionally emits blue or red light or the RGB LED, a controller 110 controls the LED light source 127 to emit which color light. Also, in the RGB LED, blue or red light is irradiated when treatment is performed, and other light (for example, green light) may be off or may be instantaneously irradiated when a power button is initially pressed. Thus, light may be used to display signals.

Since an inside of the tip 10 is vacant, when the front end portion 252 of the PCB 250 is inserted in the tip 10, the front end portion 252 needs to be fixed in the tip. Furthermore, when the inside of the tip 10 is vacant and the air is filled in the space of the tip 10, thermal conductivity of the tip 10 may be greatly lowered. In this regard, in order to fix the front end portion 252 of the PCB 250 while maintaining a performance in which heat from the heater 121 is transferred to the tip, a thermal conductive tip filling material 19 may be filled in the space of the tip 10. The thermal conductive tip filling material 19 may be a thermal conductive silicon material, and hot melt, a thermal conductive epoxy resin, or the like may be used as the thermal conductive tip filling material 19.

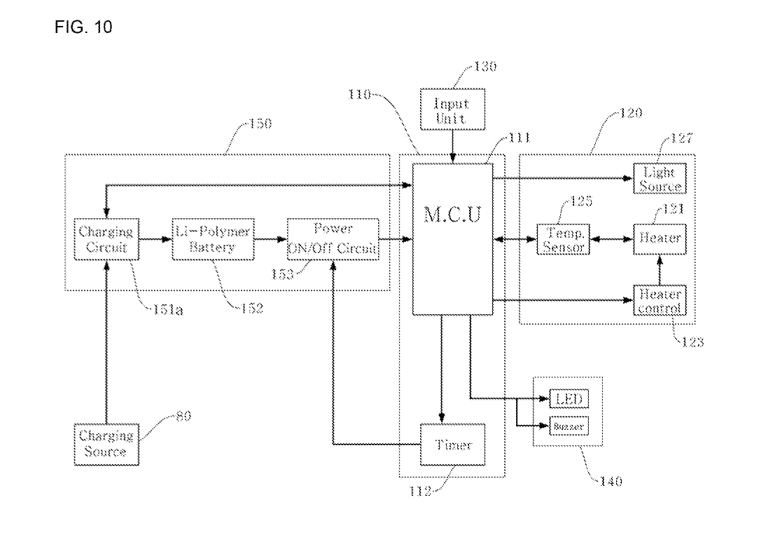

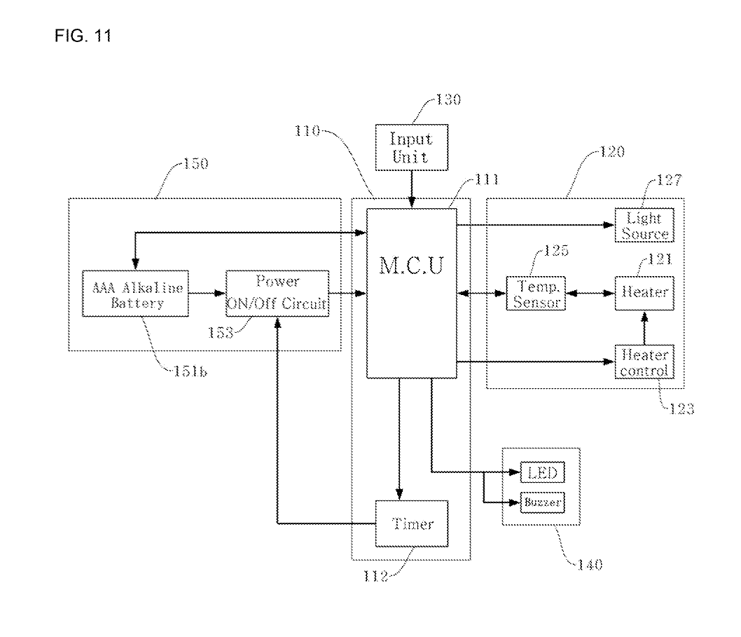

FIGS. 10 and 11 illustrate a configuration example of an internal circuit of the portable skin treatment device illustrated in FIGS. 1 and 2 and FIGS. 5 and 6. FIG. 10 corresponds to an internal configuration of the portable skin treatment device 100 illustrated in FIGS. 1 and 2, and FIG. 11 corresponds to an internal configuration of the portable skin treatment device 100 illustrated in FIGS. 5 and 6.

The internal circuit of the portable skin treatment device 100 may include five portions, i.e., a controller 110, an input unit 130, an operating unit 120, a display unit 140, and a power supply unit 150. The controller 110 controls the entire operation of the portable skin treatment device 100, and the input unit 130 transmits an input command for controlling the operation of the controller 110. The input unit 130 is a control button (50 of FIG. 7) installed on the housing of the portable skin treatment device 100. The input command may be transmitted by pressing a plurality of buttons installed to be physically differentiated from one another. Also, by using one control button 50, programming may be performed so that an input signal is interpreted and different commands are transmitted at the time when the control button 50 is pressed.

The operating unit 120 performs light irradiation treatment and thermal treatment on the user's skin, and the display unit 140 displays the state of the operating unit 120 and the operating state of the power supply unit 150 to the user. The green lamp 20 and the blue lamp 30 described above constitute an LED of the display unit 140, and a buzzer may generate a buzzing sound.

Referring to FIG. 10, the power supply unit 150 includes a charging circuit 151a that supplies power to a circuit element using an external power supply 80 as a charging source, a lithium (LI)-polymer battery 152 charged by the charging circuit 151a, and a power controller 153 that controls power on or off. The power supply unit 150 of FIG. 11 supplies power of an alkaline battery 151b (60 of FIG. 7) of 3 VA, maximum.

The controller 110 performs a control operation by using a chip of a microcomputer 111 having a built-in firmware. The controller 110 controls an input/output operation, an operation of the display unit 140, and the operation of the operating unit 120. Also, if a predetermined amount of time set by a timer circuit 112 has been elapsed, the controller 110 controls the internal circuit of the portable skin treatment device 100 so as to cut off a supply of power. For example, when the operating time of the operating unit 120 is set to 2 min 30 sec, counting is performed at the time when heating is completed, and if 2 min 30 sec has been elapsed, a supply of power is cut off

The operating unit 120 performs two skin treatments.

In light irradiation treatment, the light source 127 irradiates light onto the skin. The light source 127 may be a blue LED. Blue light emitted from the blue LED having a wavelength of 400 to 480 nm is applied to the skin so that skin treatment effects are achieved. Also, heat treatment may be performed by heating the heater 121 formed of a resistor to increase the temperature of the heater 121 to the temperature of the tip 10 of 47.2 to 49.4□ and to make the tip 10 contact the skin. A heating system may maintain the temperature of the heater 121 within the set temperature range and may be controlled by a heater controller 123 formed of a thermistor chip and a temperature sensor 125.

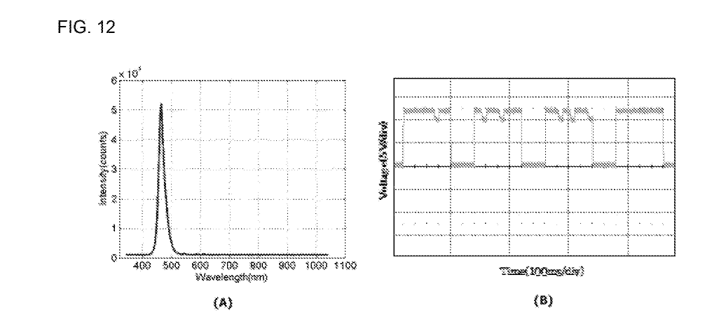

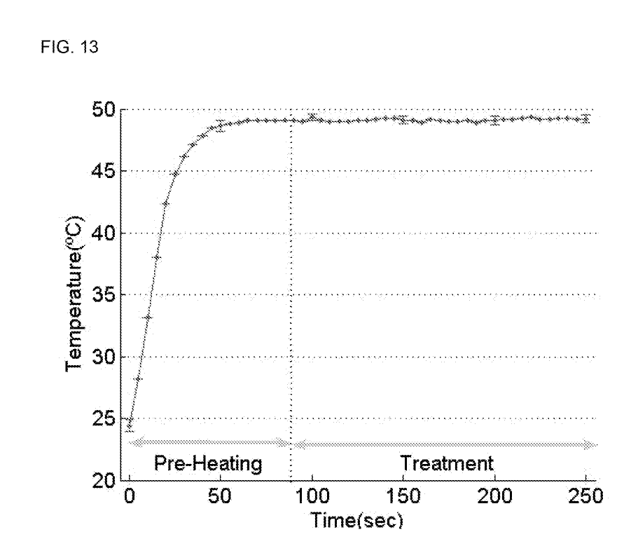

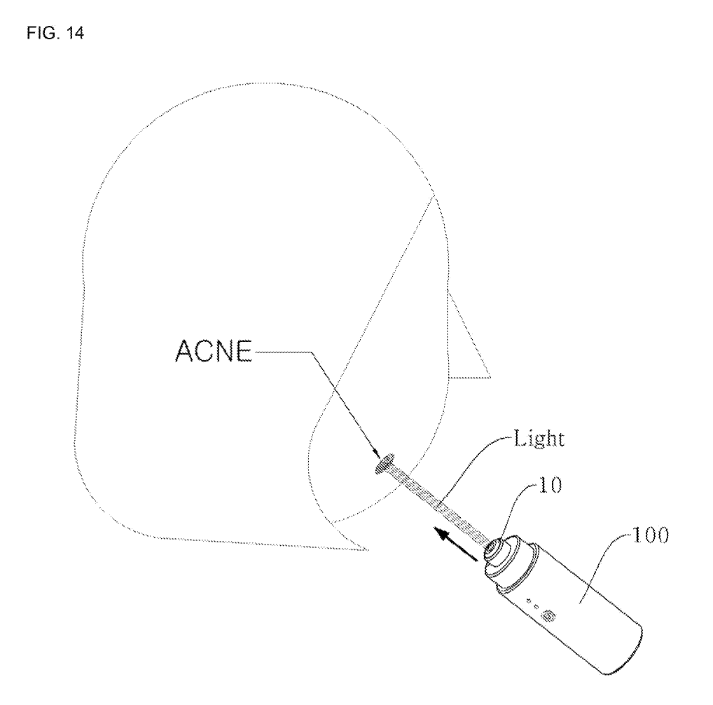

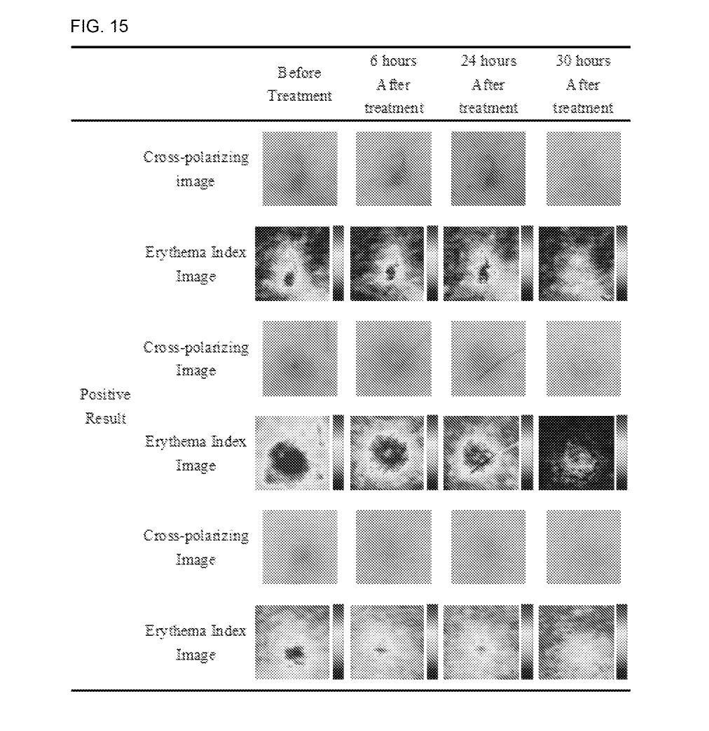

### Treatment Embodiment Using Portable Skin Treatment Device 100

<1> In the current embodiment of the present invention, a high-brightness blue LED having a single wavelength of 470 nm was used as a light source. Also, blue light having the wavelength of 470 nm was irradiated while providing a periodic peak of 5 V, as illustrated in FIGS. 12A and 12B.

<2> In heat treatment, the operating temperature of the portable skin treatment device 100 was set to 47.2 to 49.4° C. The heater 121 was preheated at the set temperature. It took about 65 seconds to reach 47.2° C. This is the same as in FIG. 13. Heated heat was applied to the skin for 2 min 30 sec, and the temperature of the heater 121 was controlled so that the range of an output temperature is 47.2 to 49.4° C.

<3> Heat treatment and light irradiation treatment were simultaneously performed in the state where the portable skin treatment device 100 contacts the skin area of a skin disease such as acne, as illustrated in FIG. 4, and the termination time was the same.

<4> As a result, treatment effects illustrated in FIG. 15 were shown.

### <Method of Controlling Operation of Portable Skin Treatment Device 100>

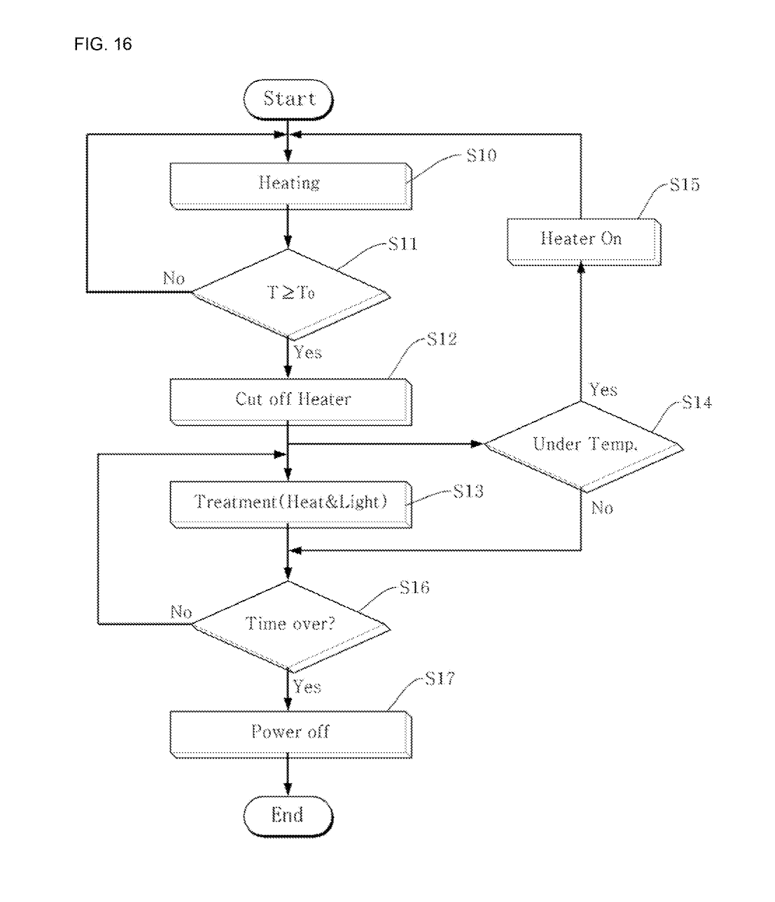

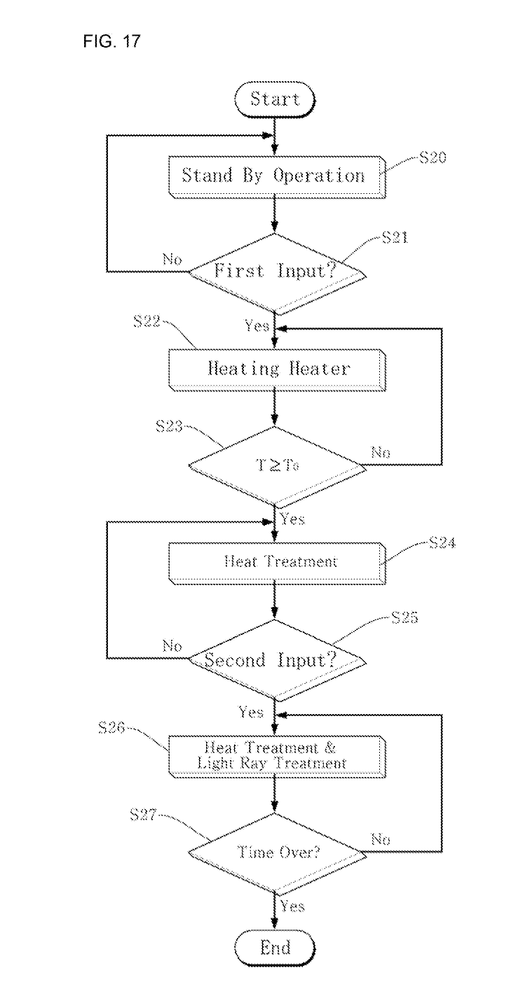

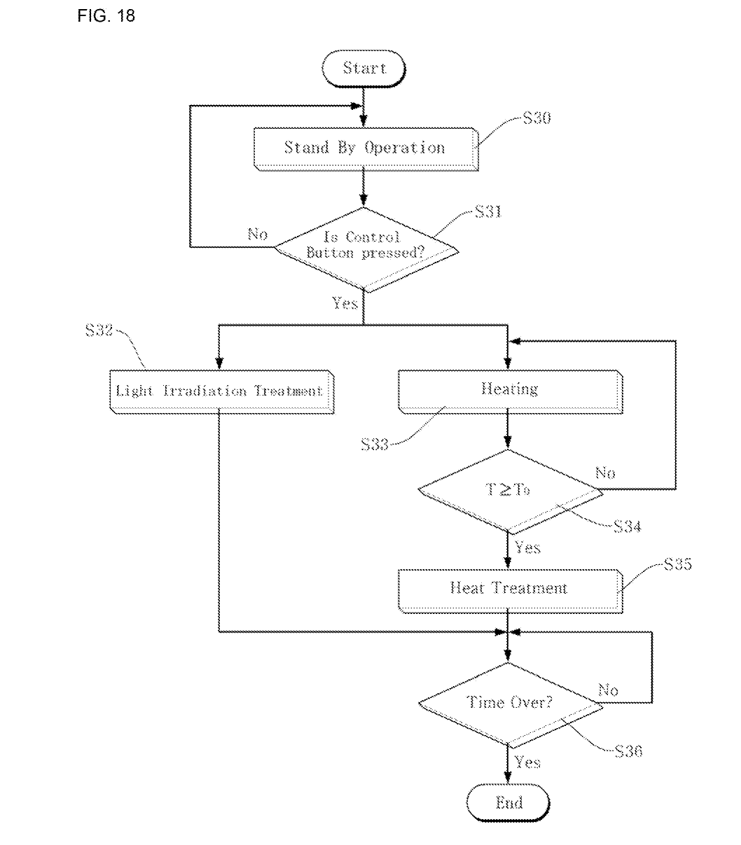

FIGS. 16 through 18 illustrate a method of controlling an operation of the portable skin treatment device 100, according to embodiments of the present invention. FIG. 16 illustrates an embodiment in which the portable skin treatment device 100 automatically performs a treatment operation by using a preset program, and FIG. 17 illustrates an embodiment in which a treatment operation is performed by an input event occurrence caused by an input unit 130, and FIG. 18 illustrates an embodiment in, after light irradiation treatment is first performed, a heater 121 is controlled to simultaneously perform heat treatment and light irradiation treatment.

The embodiment of FIG. 16 will now be described. In operation S10, if a user presses a control button 50 installed on a housing in a standby state, heating starts. Here, heating may be displayed to the user by generating a buzzing sound to turn on a green lamp 20. The portable skin treatment device 100 heats the heater 121 until the temperature of the tip 10 stably reaches a predetermined temperature range, for example, 47.2 to 49.4° C. A reference temperature T₀ at the time when an operation starts may be set to 47.2° C. In operation S11, if it is determined whether an internal temperature T of the tip 10 sensed by a temperature sensor reaches T₀ and if T=T₀ as a result of determination, in operation S12, heating by the heater 121 stops, and in operation S13, the temperature of the tip 10 is controlled to be maintained at the set temperature range, and simultaneously, a light source disposed inside the tip 10 is turned on. If the tip 10 contacts the skin, heat may be transferred to the skin through the tip 10, and simultaneously, light emitted from the light source may be irradiated onto the skin. In this regard, light irradiated onto the skin may be blue light.

The user may not contact the tip 10 the skin while the heater 121 is heated. Thus, in order to efficiently inform the user of the time when the tip 10 contacts the skin, i.e., the time when heating is completed and treatment starts, the buzzing sound may be generated to sequentially turn on/off a green lamp 20 and a blue lamp 30. Then, the blue lamp 30 is turned on after several seconds have been elapsed, it may be more efficiently informed to the user that blue light is being irradiated onto the skin.

A heater controller 123 controls a heating operation of the heater 121 so that the temperature of the heater 121 is out of the set range. In operation S14, if it is determined whether the temperature of the tip 10 falls under the reference temperature T₀ and if the temperature of the tip 10 falls under the reference temperature T₀, in operation S15, the operation of heating the heater 121 is performed again.

Since heat is applied to the skin in an artificial manner, it is not good to a human body to apply heat to the skin for a long time. Thus, an operating time needs to be set. Treatment may be terminated within the range of about 120 to 180 sec by automatically measuring time from the time when the heating operation stops and treatment starts.

The embodiment of FIG. 17 will now be described.

In operation S20, an operation stands by in a power off state so as to remove power consumption of a self-contained battery when a control button 50 is not pressed. In operation S21, it is determined whether the control button 50 installed on a housing is pressed or not. If an input signal is transmitted to a controller 110, the controller 110 controls a heater 121 to start heating, and the heater 121 starts heating in operation S22. Here, heating may be displayed to the user by generating a buzzing sound to turn on a green lamp 20. The portable skin treatment device 100 heats the heater 121 until the temperature of the tip 10 stably reaches a predetermined temperature range, for example, 47.2 to 49.4° C. A reference temperature T₀ at the time when an operation starts may be set to 47.2° C.

In operation S23, if it is determined whether an internal temperature T of the tip 10 sensed by a temperature sensor reaches T₀ and if T=T₀ as a result of determination, in operation S24, heating by the heater 121 stops, and the temperature of the tip 10 is controlled to be maintained at the set temperature range, and simultaneously, heat treatment starts. In order to efficiently inform the user of the time when heating is completed and treatment starts, the buzzing sound may be generated to sequentially turn on/off a green lamp 20 and a blue lamp 30. Until now, there is no substantial difference between the embodiment of FIG. 16 and the embodiment of FIG. 17.

However, in operation S25, a user's second input is waited without immediately performing light irradiation treatment. If the user's second input is performed through the control button 50, the controller 110 turns on a light source inside a tip 10 in addition to heat treatment being currently performed and thereby, light irradiation treatment starts. Heat may be transferred to the skin through the tip 10 that contacts the skin, and simultaneously, light emitted from the light source may be irradiated onto the skin. Here, light irradiated onto the skin may be blue light, like in the embodiment of FIG. 16. If the second input starts, the buzzing sound is generated, and the blue lamp 30 is turned on and thereby, it may be more efficiently informed to the user that blue light is being irradiated onto the skin.

In the embodiment of FIG. 17, heat treatment is performed prior to light irradiation treatment. However, heat treatment in operation S24 is performed after operation S25 and thereby operation S24 is combined with operation S26 so that heat treatment and light irradiation treatment are simultaneously performed only after the second input is performed. Here, the blue lamp 30 may be turned on. In a modified embodiment, even when a predetermined amount of time has been elapsed after the buzzing sound indicating that heating is completed is generated and if the input event does not occur, the controller 110 may control the portable skin treatment device 100 to cut off a supply of power and to return to the operation standby state. Here, the predetermined amount of time may be set to 60 sec.

Also, treatment may be terminated within the range of about 120 to 180 sec by automatically measuring time from the time when the heating operation stops and treatment starts.

The embodiment of FIG. 18 will now be described.

In operation S31, if a control button 50 is pressed in an operation standby state in operation S30, a controller 110 turns on a light source inside a tip 10, thereby starting light irradiation treatment in operation S32 and simultaneously heating a heater in operation S33. In operation S34, if it is determined whether an internal temperature T of the tip 10 sensed by a temperature sensor reaches T₀ and if T=T₀ as a result of determination, in operation S35, heating by the heater 121 stops, and the temperature of the tip 10 is controlled to be maintained at the set temperature range, and simultaneously, heat treatment starts.

In order to efficiently inform the user of the time when heating is completed and treatment starts, a buzzing sound may be generated to sequentially turn on/off a green lamp 20 and a blue lamp 30. The controller 110 controls the portable skin treatment device 100 to stop sequential turn on/off after several seconds and to turn on only the blue lamp 30. Also, like in the embodiment of FIG. 17, treatment may be terminated within the range of about 120 to 180 sec by automatically measuring time from the time when the heating operation stops and treatment starts.

In the current embodiment, heating is performed while light irradiation treatment in operation S32 is performed, and heat treatment in operation S35 and light irradiation treatment S32 are simultaneously performed from the time when heating is completed. Thus, the time when light irradiation treatment is performed is longer than the time when heat treatment is performed, so that the time when P. acne is destroyed by an active oxygen is secured by blue light. Furthermore, unlike heat, blue light does not cause a burn and is harmless to the human body and thus, there is no limitation in an irradiation time.

As described above, blue light is used during light irradiation treatment, and a high-brightness blue LED is used as a light source. However, light to be used in light irradiation treatment is not limited to blue light. Blue light is best absorbed in porphylin that is metabolite of inflammatory pimple P-acne virus and causes generation of an active oxygen, and the generated active oxygen destroys P-acne virus, and thereby, treatment effects are improved. However, a red LED may also be used as the light source to emit red light. It is known that red light has anti-inflammatory effects. In this way, blue or red light is irradiated onto the skin while heat is applied to the skin, so that improved treatment effects are shown compared to treatment effects that may be achieved when only heat treatment is performed, in related art.

As described above, skin diseases such as itch such as athlete's foot, itch in a case where a man is bitten by the insect such as mosquito, acne, and the like can be efficiently treated in any place any time.

Furthermore, improved treatment effects compared to those of a related art heat applying method can be achieved by irradiating light in a predetermined wavelength range onto the area of the skin disease as well as heat treatment.

Furthermore, invisible heat treatment and visible light treatment are simultaneously performed so that a user can easily check an operation of a portable skin treatment device and the user can do treatment for himself/herself on site immediately.

Furthermore, since an alkaline battery having the specification of AAA or AA that the user can easily purchase is inserted in or removed from the portable skin treatment device, inconvenience that occurs during the use of a battery can be avoided.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of controlling an operation of a portable skin treatment device, the method comprising:
heating a heater to prepare heating of a tip of the treatment device in a predetermined temperature range, if a control button of the treatment device is pressed; and
turning on a light source disposed inside a housing of the treatment device and performing light irradiation treatment of irradiating light onto the skin through the tip together with heat, if heating of the tip is completed,
wherein the light source is a blue light emitting diode (LED), a red LED, an LED having red, green, and blue (RGB) color, or an LED that optionally emits blue or red light, and
when the light irradiation treatment is performed together with heat, a temperature of the tip is 47.2 to 49.4° C., and the time when the light irradiation treatment is performed together with heat is 120 to 180 sec.

2. A portable skin treatment device that operates by using a method of claim 1, the portable skin treatment device including:
a housing which comprises a control button, a display unit, and a tip for skin contact and in which a printed circuit board (PCB) is disposed,
wherein internal circuit elements are mounted on the PCB, the internal circuit elements comprising:
a heater heating the tip and a heater controller controlling the heater;
a light source irradiating light onto an outside of the treatment device through the tip;
a microcomputer controller controlling an operation of the treatment device; and
a power supply unit supplying power, and
wherein the light source is attached to an edge surface of an end of the PCB that contacts the tip.

3. The portable skin treatment device of claim 2, wherein the tip has a hollow cylindrical shape, and the light source is disposed in an internal space of the tip, and a thermal conductive filling material is filled in the light source and the internal space of the tip.

4. A method of controlling an operation of a portable skin treatment device, the method comprising:
determining an input of a control button of the treatment device;
turning on a light source disposed inside a housing of the treatment device and irradiating light onto the skin through the tip together with heat, if the input is performed;
heating a heater so that a temperature of a tip of the treatment device reaches a predetermined temperature, while the light irradiation treatment is performed; and
outputting a heating completion signal to a display unit of the treatment device if heating is completed, and performing heat treatment in addition to the light irradiation treatment,
wherein the light source is a blue light emitting diode (LED), a red LED, an LED having red, green, and blue (RGB) color, or an LED that optionally emits blue or red light, and
when the light irradiation treatment is performed together with the heat treatment, a temperature of the tip is 47.2 to 49.4° C., and the time when the light irradiation treatment is performed together with the heat treatment is 120 to 180 sec.

5. A portable skin treatment device that operates by using a method of claim 4, the portable skin treatment device including:
a housing which comprises a control button, a display unit, and a tip for skin contact and in which a printed circuit board (PCB) is disposed,
wherein internal circuit elements are mounted on the PCB, the internal circuit elements comprising:
a heater heating the tip and a heater controller controlling the heater;
a light source irradiating light onto an outside of the treatment device through the tip;
a microcomputer controller controlling an operation of the treatment device; and
a power supply unit supplying power, and
wherein the light source is attached to an edge surface of an end of the PCB that contacts the tip.

6. The portable skin treatment device of claim 5, wherein the tip has a hollow cylindrical shape, and the light source is disposed in an internal space of the tip, and a thermal conductive filling material is filled in the light source and the internal space of the tip.

7. A portable skin treatment device in which a printed circuit board (PCB) on which an internal circuit unit is mounted, is inserted in a housing having a hollow tube shape, the portable skin treatment device comprising:
a tip for skin contact installed on one end of the housing;
a battery cap installed on the other end of the housing, sealing the housing and simultaneously electrically contacting a terminal inside the housing;
a battery unit in which two alkaline batteries having a specification of AAA or AA and an electromotive force of 1.5 V are inserted in the housing; and
a control button and a display unit installed on a surface of the housing, wherein internal circuit elements are mounted on the PCB, the internal circuit elements comprising:
a heater heating the tip and a heater controller controlling the heater;
a light source irradiating light being a light emitting diode (LED) attached to an edge surface of the end of the PCB that contacts the tip and irradiating light onto an outside of the treatment device through the tip; and
a microcomputer controller receiving an input of the control button, controlling an operation of the treatment device and displaying an operating state on the display unit,
wherein heat treatment and light irradiation treatment are performed by the input of the control button.

8. The portable skin treatment device of claim 7, wherein the tip has a hollow cylindrical shape, and the light source is disposed in an internal space of the tip, and a thermal conductive filling material is filled in the light source and the internal space of the tip.
